(19) United States
(12) Patent Application Publication
(10) Pub. No.: US 2023/0036503 A1
(43) Pub. Date: Feb. 2, 2023

(54) Fusion Protein, Amino Acid Sequence Thereof, Coding Nucleotide Sequence Thereof, Preparation Method Thereof and Use Thereof

(71) Applicants: SICHUAN AGRICULTURAL UNIVERSITY, Ya'an, Sichuan (CN); CHENGDU LUSYNO BIOTECHNOLOGY CO., LTD, Chengdu, Sichuan (CN)

(72) Inventors: Yi CAI, Chengdu, Sichuan (CN); Qi LI, Chengdu, Sichuan (CN); Jinya GUO, Chengdu, Sichuan (CN); Yong LI, Chengdu, Sichuan (CN); Pingping YANG, Chengdu, Sichuan (CN); Xinqiong ZHOU, Chengdu, Sichuan (CN); Limei ZHANG, Chengdu, Sichuan (CN); Huaiyu ZHANG, Chengdu, Sichuan (CN); Xiangrong WANG, Chengdu, Sichuan (CN)

(21) Appl. No.: 17/757,280

(22) PCT Filed: Dec. 10, 2020
(86) PCT No.: PCT/CN2020/135367

§ 371 Date: Jun. 13, 2022

(30) Foreign Application Priority Data
Dec. 11, 2019 (CN) 201911265176.9

(51) Int. Cl. C07K 14/415; C07K 14/195; C12N 15/62; C12N 15/82

(52) U.S. Cl. CPC C07K 14/415; C07K 14/195; C12N 15/62; C12N 15/82; C07K 2319/21

(57) ABSTRACT

A fusion protein, an amino acid sequence thereof, a coding nucleotide sequence thereof, a preparation method thereof and a use thereof are in the technical field of agricultural biotechnology. The fusion protein contains or consists of at least three, four, five, six, seven, or eight same and/or different PAMP (Pathogen-Associated Molecular Pattern) polypeptides. Optionally, there is at least one linker or no linker between two adjacent PAMP polypeptides. A plurality of PAMP polypeptides are assembled into the fusion protein having multiple immune epitopes. The fusion protein may induce defense immune responses of plants, weaken infestation ability of pathogenic microorganisms and substantially improve the disease resistance of plants. The method for preparing the fusion protein combines technologies of PTI (PAMP-Triggered Immunity) mechanism and gene engineering to obtain the fusion protein having multiple immune epitopes can be used in preparation of plant immune PAMP polypeptides.

### INCORPORATION OF SEQUENCE LISTING

This application contains a sequence listing submitted in Computer Readable Form (CRF). The CFR file containing the sequence listing entitled “PA288-0106_ST25.txt”, which was created on Jun. 9, 2022, and is 12,409 bytes in size. The information in the sequence listing is incorporated herein by reference in its entirety.

### TECHNICAL FIELD

The present disclosure relates to the technical field of agricultural biotechnology, and in particular, to a fusion protein, an amino acid sequence thereof, a coding nucleotide sequence thereof, a preparation method thereof, and a use thereof.

### BACKGROUND ART

In the agroecological system, compared with a chemical pesticide, a bio-pesticide product has advantages of low toxicity, short degradation period, high environment compatibility and the like, for which it has already been widely applied to crop production. However, conventional researches and developments on bio-pesticides mainly focus on screening organism-derived antibacterial and pesticidal substances to develop bio-pesticides to resist diseases and insect pests, and often ignore the intrinsic immunity of plants in resisting diseases and the insect pests. In recent years, it has been increasingly noticed on plant immune induction technologies in development of the bio-pesticides. A plant immune inducer, also referred to as a “plant vaccine”, stimulates intrinsic immune system of plants to resist diseases, and increase yield and quality. Compared with conventional bio-pesticides, the “plant vaccine” cause the no resistance of pathogenic microorganisms, making it more suitable for green and healthy agricultural production, and having already attracting wide attention at home and aboard.

The PTI (PAMP-triggered immunity) mechanism of the plants has a wide prospect in development on bio-pesticides. At present, there are few types of plant immune pesticides only on the market, including Atailing, harpin proteins and other several types of plant immune pesticides. However, these products have considerable limitations. Firstly, with still unclear receptors, it is difficult to make a scientific and effective guidance for usage thereof, making determination of applications and administration concentrations only possible through experimental experiences. Secondly, as plants have low sensitivity to these plant immune pesticides, these plant immune pesticides must be used in high concentrations, resulting in high costs. An Axiom Harpin Protein is a biological protein pesticide relatively widely used internationally. However, a product containing 1% Axiom Harpin Protein is sold at a price higher than 140 RMB per gram which is over 5000 RMB per gram pure protein, imposing great limitation on application of the harpin protein in agricultural production. Thus, it is a desirable for an effective bio-pesticide product that is low in cost and enhances the intrinsic immunity of plants.

### SUMMARY OF THE INVENTION

In view of this, the present disclosure provides a fusion protein, in which a plurality of PAMP (Pathogen-Associated Molecular Pattern) polypeptides are assembled into the fusion protein having one or more immune epitopes. The fusion protein may more rapidly and widely bind to receptors on surfaces of plant cells, induce immune responses of various plants and obviously improve the disease resistance of plants. Compared with PAMP polypeptides, the fusion protein has a lower preparation cost, and increased unit yield of protein bio-pesticide products, reducing an expected cost of proteins to 1 RMB/g, resulting in low application concentration and being effective rapidly. Therefore, applications thereof in agricultural production may substantially lower the production cost.

In a first aspect, the present disclosure provides a fusion protein containing or consisting of at least three, four, five, six and seven same and/or different PAMP polypeptides. Optionally, there is at least one linker or no linker between two adjacent PAMP polypeptides.

Pathogen-associated molecular patterns (PAMPs) refer to some highly conserved molecules in evolution, characterized by rapidly triggering immune defense responses of plants. There are diverse types of PAMP molecules, including polysaccharides, lipids, polypeptides and other molecular substances. Polypeptide PAMPs are referred to as the PAMP polypeptides. Most PAMPs are essential in the life cycle of pathogenic bacteria and are usually used by plants as signaling molecules for perceiving invasion of pathogenic bacteria. Researches show that, with interaction between specific PAMP polypeptides and receptor proteins on cell membranes of plants, the defense responses of plants may be rapidly triggered to resist infection of pathogenic bacteria. Defense immune responses of plants include: inducing rapid generation of reactive oxygen species (ROS) in plants and deposition of callose; synthesizing a large amount of phytohormones (for example, salicylic acid and ethylene); and rapidly expressing defense genes. Here, certain autocrine peptides (such as PIP1 and PEP1) that are produced by plants being stimulated, also referred to as PAMP polypeptides, may serve as a second messenger for enhancing immune signals and keeping a persistent immune effect.

The PAMP polypeptide is typically obtained by finding a relatively conserved sequence in protein molecules through comparative analysis on homologous sequences in various pathogenic fungi or bacteria, and then artificially synthesizing the polypeptide sequence and verifying ability thereof to activate the immune responses of plants.

Due to different types of pathogenic bacteria and naturally occurring mutations, there are various natural mutants of the PAMP polypeptide; and mutants of the PAMP polypeptide may keep an immunogenicity greater than or equal to 80% of that of a wild-type PAMP polypeptide and can activate a same and fixed immune receptor as the PAMP polypeptide.

It can be understood that “the fusion protein contains or consists of at least three, four, five, six and seven same and/or different PAMP polypeptides” may be interpreted as:

(1) the fusion protein contains or consists of at least three, four, five, six and seven same types of PAMP polypeptides, that is, including, but not limited to, three, four, five, six, seven or more same types of PAMP polypeptides;

(2) the fusion protein contains or consists of at least three, four, five, six and seven different types of PAMP polypeptides, that is, including, but not limited to, three, four, five, six, seven or more different types of PAMP polypeptides; and

(3) the fusion protein contains or consists of at least three, four, five, six and seven same and different types of PAMP polypeptides, that is, including, but not limited to, two same plus one different, two same plus two different, two same plus three different, two same plus four different, two same plus five different, two same plus six different, two same plus seven different or other more same and different types of PAMP polypeptides.

“Optionally” represents may be or may not be. In other words, there may be no linker or at least one linker between two adjacent PAMP polypeptides constituting the fusion protein. The linker is a linkage region having one or more amino acid residues, preferably, at least three consecutive amino acid residues. The two adjacent PAMP polypeptides are linked through the linker, and then at least three polypeptides are assembled into the fusion protein.

The linker includes, but is not limited to, GAG, AGA, AAA, GGG, KRK, KKK, RRR and AKG.

It should be noted that an arrangement of the PAMP polypeptides constituting the fusion protein and specific types of the linker are not specifically defined herein, and proper variations, substitutions and adjustments may be made according to types of plants or types of targeting pathogenic microorganisms, as along as immune resistance of plants may be induced.

By researching the PTI (PAMP-Triggered Immunity) mechanism of plants, by virtue of the gene engineering technology, the present disclosure assembles at least three specific PAMP polypeptides into the fusion protein through a molecular design and directed assembly. With one or more immune epitopes, the fusion protein may more rapidly and widely bind to the receptors on surfaces of plant cells, induce defense immune responses of plants, weaken infection ability of pathogenic microorganisms and substantially improve disease resistance of plants. Moreover, serving as a bio-pesticide product, the fusion protein further has advantages of low toxicity, short degradation period, high environment compatibility, resulting in no insecticide tolerance of pathogenic bacteria and the like.

In an embodiment of the present disclosure, the fusion protein mainly consists of at least three, four, five, six and seven same and/or different PAMP (Pathogen-Associated Molecular Pattern) polypeptides; and there is at least one linker or no linker between the two adjacent PAMP polypeptides. Furthermore, the PAMP polypeptides includes a first polypeptide for activating an immune receptor FLS2, a second polypeptide for activating an immune receptor RLP23, a third polypeptide for activating an immune receptor EFR, a fourth polypeptide for activating an immune receptor RLK7, a fifth polypeptide for activating an immune receptor PEPR1, a sixth polypeptide for activating an immune receptor CORE1, a seventh polypeptide for activating an immune receptor FLS3, an eighth polypeptide for activating an immune receptor FER, a ninth polypeptides pep13 for activating immune response of plants, a tenth polypeptides hrp24 for activating immune responses of plants and an eleventh polypeptides sys18 for activating the immune responses of the plants.

The fusion protein contains various polypeptide components for activating different receptors, providing many advantages. Firstly, various signaling pathways may activate immune responses of different plants, avoiding failure of generating immune responses due to lack of particular receptors in certain plants, and making the fusion protein adaptive to various plants. Secondly, when various signaling pathways activate a same immune response of plants at the same time, generated immune signals may have synergistic effect, resulting in more sensitive and efficient immune activation.

Furthermore, the first polypeptide for activating the immune receptor FLS2 is flg15 and homoeotic mutants thereof or flg22 and homoeotic mutants thereof.

An amino acid sequence of Flg15 is shown as SEQ ID NO: 1: RINSAKDDAAGLQIA. The homoeotic mutants of flg15 include PAMP polypeptides having one or more (for example, 1-10) amino acids added, deleted or substituted in the amino acid sequence shown as SEQ ID NO: 1 and capability of activating the immune receptor FLS2.

Flg22 is a very highly conserved region at N terminal of bacterial flagellin. Many researches have proved that flg22 may induce intrinsic immunity of plants, activate the receptor FLS2, and act on signaling pathways such as SA signaling pathway and MAPK signaling pathway, delivering an important influence on disease resistance of plants. An amino acid sequence of flg22 is shown as SEQ ID NO: 2: QRLSTGSRINSAKDDAAGLQIA. The homoeotic mutants of flg22 include polypeptides having one or more (for example, 1-10) amino acids added, deleted or substituted in the amino acid sequence shown as SEQ ID NO: 2 and the ability of activating the immune receptor FLS2.

Furthermore, the first polypeptide for activating the immune receptor FLS2 is, preferably, flg22 and homoeotic mutants thereof. Specifically, a homoeotic mutant flg22ₘ₁ of flg22 generates substitution mutations at 4 amino acids in the amino acid sequence shown as SEQ ID NO: 2 at located positions 1, 5, 7 and 8: Q1T, T5 S, S7L and R8K; and a homoeotic mutant flg22ₘ₂ of flg22 generates substitution mutations at 4 amino acids in the amino acid sequence shown as SEQ ID NO: 2 located at positions 5, 7, 20 and 22: T5 S, S7L, Q20A and A22S.

The above first polypeptides having different amino acid sequences all belong to homoeotic mutants having a same function, and may all activate the immune receptor FLS2. As such, they are proved to have similar bioactivities. For details, refer to introductions for the polypeptides for activating the immune receptor FLS2 in the literature “Plants have a sensitive perception system for the most conserved domain of bacterial flagellin” and Paper “CD2-1, the C-Terminal Region of Flagellin, Modulates the Induction of Immune Responses in Rice”.

Furthermore, the second polypeptide for activating the immune receptor RLP23 is preferably polypeptide nlp20 and homoeotic mutants thereof.

Nlp20 is a polypeptide molecule consisting of 20 amino acids and a characteristic amino acid sequence contained in Necrosis- and ethyleninducing peptide-like proteins (NLPs). Researches have showed that the polypeptides nlp20 may rapidly cause immune responses of plants and enhance immunity of plants to infection of microorganisms by activating an immune recognition receptor RLP23.

An amino acid sequence of nlp20 is shown as SEQ ID NO: 3: AIMYSWYFPKDSPVTGLGHR. The homoeotic mutants of nlp20 include PAMP polypeptides with one or more (for example, 1-10) amino acids added, deleted or substituted in the amino acid sequence shown as SEQ ID NO: 3, and an ability to activate the immune receptor RLP23. Specifically, a homoeotic mutant nlp20ₘ₁ of nlp20 generates substitution mutations at 3 amino acids in the amino acid sequence shown as SEQ ID NO: 3 located at positions 8, 14 and 17: F8M, V145 and L171; and a homoeotic mutant nlp20ₘ₂ of nlp20 generates substitution mutations at 3 amino acids in the amino acid sequence shown as SEQ ID NO: 3 located at positions 5, 14 and 15: S5A, V145 and T15P.

The above second polypeptides having different amino acid sequences all belong to homoeotic mutants having a same function, and may all activate the immune receptor RLP23. As such, they are proved to have similar bioactivities. For details, refer to introductions for the polypeptides for activating the immune receptor RLP23 in the literature “A Conserved Peptide Pattern from a Widespread Microbial Virulence Factor Triggers Pattern-Induced Immunity in Arabidopsis”.

Furthermore, the third polypeptide for activating the immune receptor EFR is preferably a polypeptide elf18 and homoeotic mutants thereof.

Elf18 is a polypeptide having 18 amino acids at the N terminal of bacterial protein elongation factor Tu (EF-Tu). It may activate a receptor EFR, induce oxidative burst and biosynthesis of ethylene, and triggers resistance to subsequent infection of pathogenic bacteria.

An amino acid sequence of elf18 is shown as SEQ ID NO: 4: SKEKFERTKPHVNVGTIG. The homoeotic mutants of elf18 include PAMP polypeptides with one or more (for example, 1-10) amino acids added, deleted or substituted in the amino acid sequence shown as SEQ ID NO: 4 and the ability to activate the immune receptor EFR. Specifically, a homoeotic mutant elf18ₘ₁ of elf18 generates substitution mutations at 4 amino acids in the amino acid sequence shown as SEQ ID NO: 4 located at positions 1, 3, 8 and 14: S1A, E3S, T8N and V14I; and a homoeotic mutant elf18ₘ₂ of elf18 generates substitution mutations at 5 amino acids in the amino acid sequence shown as SEQ ID NO: 4 located at positions 1, 6, 8, 9 and 12: S1V, E6D, T8 S, K9L and V12C.

The above third polypeptides having different amino acid sequences all belong to homoeotic mutants having a same function, and may all activate the immune receptor EFR. As such, they are proved to have similar bioactivities. For details, refer to the introduction for the polypeptides for activating the immune receptor EFR in the literature “The N Terminus of Bacterial Elongation Factor Tu Elicits Innate Immunity in Arabidopsis Plants”.

Furthermore, the fourth polypeptide for activating the immune receptor RLK7 is preferably a polypeptide pip1 and homoeotic mutants thereof.

Pip1 is a polypeptide having 13 amino acids, secreted by prePIP1 to an extracellular space surrounding cells and subjected to cleavage of amino acids from the conserved C-terminal region. Pip1 activates the receptor RLK7 through a signal sent by a receptor-like kinase 7 (RLK7) on surfaces of cells, thereby activating immune responses of plants and enhancing resistance of plants to pathogens.

An amino acid sequence of pip1 is shown as SEQ ID NO: 5: RLASGPSPRGPGH. The homoeotic mutants of pip1 include PAMP polypeptides with one or more (for example, 1-10) amino acids added, deleted or substituted in the amino acid sequence shown as SEQ ID NO: 5 and the ability of activating the immune receptor RLK7. Specifically, the homoeotic mutant pip1ₘ₁ of pip1 adds of 2 amino acids FV between positions 1 and 2 in the amino acid sequence shown as SEQ ID NO: 5 and generates substitution mutations at 3 amino acids at positions 2, 3 and 9.

The above fourth polypeptides having different amino acid sequences all belong to homoeotic mutants having a same function, and may all activate the immune receptor RLK7. As such, they are proved to have similar bioactivities. For details, refer to the introduction for the polypeptides for activating the immune receptor RLK7 in the literature “The Secreted Peptide PIP1 Amplifies Immunity through Receptor-Like Kinase 7”.

Furthermore, the fifth polypeptide for activating the immune receptor PEPR1 is preferably polypeptide pep1 and homoeotic mutants thereof.

Pep1 is a kind of endogenous molecules, is derived from a polypeptide comprising 23 amino acids at the C-terminal of a precursor protein proPEP1 and can activate the receptor PEPR1 and the intrinsic immunity of plants.

An amino acid sequence of pep1 is shown as SEQ ID NO: 6: ATKVKAKQRGKEKVSSGRPGQHN. The homoeotic mutants of pep1 include PAMP polypeptides with one or more (for example, 1-10) amino acids added, deleted or substituted in the amino acid sequence shown as SEQ ID NO: 6, and the ability to activate the immune receptor PEPR1. Specifically, a homoeotic mutant pep1ₘ₁ of pep1 generates substitution mutations at 9 amino acids in the amino acid sequence shown as SEQ ID NO: 6 located at positions 1, 4, 5, 6, 8, 10, 11, 12 and 13: A1E, V4A, K5R, A6G, Q8N, G10T, E11P, K12T and V13P; and a homoeotic mutant pep1ₘ₂ of pep1 deletes 8 amino acids in the amino acid sequence shown as SEQ ID NO: 6 before the position 9, and generates substitution mutations at 1 amino acid located at position 10: G10A.

The above fifth polypeptide having different amino acid sequences all belong to homoeotic mutants having a same function, and may all activate the immune receptor PEPR1. As such, they are proved to have similar bioactivities. For details, refer to the introduction for the polypeptides for activating the immune receptor PEPR1 in the literatures “Structure-activity studies of AtPep1, a plant peptide signal involved in the innate immune response” and “An endogenous peptide signal in Arabidopsis activates components of the innate immune response”.

Furthermore, the sixth polypeptide for activating the immune receptor CORE1 may be a polypeptide csp15 and homoeotic mutants thereof or polypeptide csp22 and homoeotic mutants thereof.

An amino acid sequence of csp15 is shown as SEQ ID NO: 7: VKWFNAEKGFGFITP. The homoeotic mutants of csp15 include PAMP polypeptides with one or more (for example, 1-10) amino acid added, deleted or substituted in the amino acid sequence shown as SEQ ID NO: 7 and the ability to activate the immune receptor CORE1.

Csp22 is a polypeptide having 22 amino acids on a conserved domain of a bacterial cold shock protein (CSP), which can activate the receptor CORE1 and may efficiently induce defense responses of tobaccos. An amino acid sequence of csp22 is shown as SEQ ID NO: 8: AVGTVKWFNAEKGFGFITPDDG. The homoeotic mutants of csp22 include polypeptides with one or more (for example, 1-10) amino acids added, deleted and substituted in the amino acid sequence shown as SEQ ID NO: 8 and the ability to activate the immune receptor CORE1.

Furthermore, the sixth polypeptide for activating the immune receptor CORE1 is preferably the polypeptide csp22 and the homoeotic mutants thereof. Specifically, a homoeotic mutant csp22ₘ₁ of csp22 generates substitution mutation at 1 amino acid in the amino acid sequence shown as SEQ ID NO: 8 located at position 11: E11A; and a homoeotic mutant csp22ₘ₂ of csp22 generates substitution mutation at 1 amino acid in the amino acid sequence shown as SEQ ID NO: 8 located at position 14: F14Y.

The above sixth polypeptide having different amino acid sequences all belong to homoeotic mutants having a same function, and may all activate the immune receptor CORE1. As such, they are proved to have similar bioactivities. For details, refer to the introduction for the polypeptides for activating the immune receptor CORE1 in the literature “The highly conserved RNA-binding motif RNP-1 of bacterial cold shock proteins is recognized as an elicitor signal in tobacco”.

Furthermore, the seventh polypeptide for activating the immune receptor FLS3 is preferably polypeptide flgII-28 and homoeotic mutants thereof.

Both flgII-28 and flg22 are very highly conserved regions at the N-terminal of the bacterial flagellin, which are the main PAMPs recognized by plants. FlgII-28 can activate the receptor FLS3, stimulate the plants to increase production of ethylene as a stress hormone, accelerate production of ROS and activate immune responses of plants.

An amino acid sequence of flgII-28 is shown as SEQ ID NO: 9: ESTNILQRMRELAVQSRNDSNSATDREA. The homoeotic mutants of flgII-28 include PAMP polypeptides with one or more (for example, 1-10) amino acids added, deleted or substituted in the amino acid sequence shown as SEQ ID NO: 9 and the ability to activate the immune receptor FLS3. Specifically, a homoeotic mutant flgII-28ₘ₁ of flgII-28 generates substitution mutations at 2 amino acids in the amino acid sequence shown as SEQ ID NO: 9 located at positions 23 and 27: A23S and E27D; and a homoeotic mutant flgII-28ₘ₂ of flgII-28 generates substitution mutations at 3 amino acids in the amino acid sequence shown as SEQ ID NO: 9 located at positions 13, 23 and 27: A13V, A23S and E27D.

The above seventh polypeptide having different amino acid sequences all belong to homoeotic mutants having a same function, and may all activate the immune receptor FLS3. As such, they are proved to have similar bioactivities. For details, refer to the introductions for the polypeptides for activating the immune receptor FLS3 in the literatures “Allelic variation in two distinct Pseudomonas syringae flagellin epitopes modulates the strength of plant immune responses but not bacterial motility” and “Natural Variation for Responsiveness to flg22, flgII-28, and csp22 and Pseudomonas syringae pv. tomato in Heirloom Tomatoes”.

Furthermore, the eighth polypeptide for activating the immune receptor FER is preferably polypeptide ralf17 and homoeotic mutants thereof.

An amino acid sequence of ralf17 is shown as SEQ ID NO: 10: NSIGAPAMREDLPKGCAPGSSAGCKMQPANPYKPGCEASQRCRGG. The homoeotic mutants of ralf17 include PAMP polypeptides with one or more (for example, 1-10) amino acids added, deleted or substituted in the amino acid sequence shown as SEQ ID NO: 10 and the ability to activate the immune receptor FER. Specifically, a homoeotic mutant ralf17ₘ₁ of ralf17 generates substitution mutations at 4 amino acids in the amino acid sequence shown as SEQ ID NO: 10 located at positions 1, 2, 5 and 12: N1K, S2T, A5N and L12E.

The above eighth polypeptide having different amino acid sequences all belong to homoeotic mutants having a same function, and may all activate the immune receptor FER. As such, they are proved to have similar bioactivities. For details, refer to the introductions for the polypeptides for activating the immune receptor FER in the literatures “The receptor kinase FER is a RALF-regulated scaffold controlling plant immune signaling” and “How CrRLK1L receptor complexes perceive RALF signals”. Furthermore, the PAMP polypeptides further include the ninth polypeptide for activating the immune responses of the plants, preferably, the polypeptide pep13 and homoeotic mutants thereof pep13 is a conserved polypeptide fragment in a cell wall glycoprotein GP42. The cell wall glycoprotein exists widely in oomycetes. Therefore, pep13 has an important influence on recognition of pathogens of oomycetes and activation of defense responses of plants. Particularly in parsley and potatos, pep13 may mediate expression of a defense gene and induce synthesis of antibacterial phytoalexin.

An amino acid sequence of pep13 is shown as SEQ ID NO: 11: VWNQPVRGFKVYE. The homoeotic mutants of pep13 include PAMP polypeptides with one or more (for example, 1-10) amino acids added, deleted or substituted in the amino acid sequence shown as SEQ ID NO: 11 and the ability of activate the immune responses of the plants. Specifically, a homoeotic mutant pep13ₘ₁ of pep13 generates substitution mutation at 1 amino acid in the amino acid sequence shown as SEQ ID NO: 11 located at position 12: Y12F; and a homoeotic mutant pep13ₘ₂ of pep13 generates substitution mutation at 1 amino acid in the amino acid sequence shown as SEQ ID NO: 11 located at position 12: Y12A.

The above ninth polypeptide having two different amino acid sequences all belong to homoeotic mutants having a same function and are proved to have similar bioactivities. For details, refer to the introduction in the literature “Pep-13, a plant defense-inducing pathogen associated pattern from Phytophthora transglutaminases”.

Furthermore, the PAMP polypeptide further include the tenth polypeptides for activating the immune responses of plants, preferably, the polypeptide hrp15 and homoeotic mutants thereof.

An amino acid sequence of hrp15 is shown as SEQ ID NO: 12: DLGQLLGGLLQKGLE. The homoeotic mutants of hrp15 comprise PAMP polypeptides with one or more (for example, 1-10) amino acids added, deleted or substituted in the amino acid sequence shown as SEQ ID NO: 12 and the ability to activate the immune responses of plants. Specifically, a homoeotic mutant hrp15ₘ₁ of hrp15 generates substitution mutations at 9 amino acids in the amino acid sequence shown as SEQ ID NO: 12: D1Q, G3D, G7T, G8Q, L10I, Q11M, K12A, G13L and E15Q; and a homoeotic mutant hrp24 of hrp15 adds 9 amino acids in the amino acid sequence shown as SEQ ID NO: 12.

Furthermore, the tenth polypeptide is preferably the homoeotic mutant hrp24 of the polypeptide hrp15. The homoeotic mutant hrp24 of hrp15 adds 9 amino acids in the amino acid sequence shown as SEQ ID NO: 12. An amino acid sequence of hrp24 is shown as SEQ ID NO: 13: PNQDLGQLLGGLLQKGLEATLQDA.

The above tenth polypeptide having two different amino acid sequences all belong to homoeotic mutants having a same function and are proved to have similar bioactivities. For details, refer to the introduction in the literature “Functional mapping of harpin hrpZ of Pseudomonas syringae reveals the sites responsible for protein oligomerization, lipid interactions and plant defence induction”.

Furthermore, the PAMP polypeptides further include the eleventh polypeptides for activating the immune responses of plants, preferably, the polypeptide sys18 and homoeotic mutants thereof.

An amino acid sequence of sys18 is shown as SEQ ID NO: 14: AVQSKPPSKRDPPKMQTD. The homoeotic mutants of sys18 include PAMP polypeptides with one or more (for example, 1-10) amino acids added, deleted or substituted in the amino acid sequence shown as SEQ ID NO: 14 and the ability to activate the immune responses of plants. Specifically, a homoeotic mutant sys18ₘ₁ of sys18 generates substitution mutation at 1 amino acid in the amino acid sequence shown as SEQ ID NO: 14 located at position 6: P6A; and a homoeotic mutant sys18ₘ₂ of sys18 generates substitution mutation at 1 amino acid in the amino acid sequence shown as SEQ ID NO: 14 located at position 10: R10A.

The above eleventh polypeptide having two different amino acid sequences all belong to homoeotic mutants having a same function and are proved to have similar bioactivities. For details, refer to the introduction in the literature “Structure-activity of deleted and substituted systemin.an 18-amino acid polypeptide inducer of plant defensive genes”.

It should be noted that names of all the above mutants are not their intrinsic biological names, but are uniformly named for convenience in writing and understanding of the Patent. For example, with regard to nlp20ₘ₁, the biological name is Pyanlp20, and other mutants have the same naming mode. The biological name of a specific mutant can be searched in papers or databases based on an amino acid sequence.

It should be noted that, due to naturally occurring mutations, with respect to PAMP polypeptides for activating different receptors, the PAMP polypeptides having the disclosed amino acid sequences, as well as the polypeptides mutants such as PAMP polypeptide mutants formed by adding, deleting or substituting one or more amino acid sequences in the original amino acid sequences and capable of activating the same receptors, all fall within the protection scope of the present disclosure.

In a preferred embodiment of the present disclosure, the first polypeptide is flg22; the second polypeptide is nlp20; the third polypeptide is elf18; the fourth polypeptide is pip1; the fifth polypeptide is pep1; the sixth polypeptide is csp22; the seventh polypeptide is flgII-28; the eighth polypeptide is ralf17; the ninth polypeptide is pep13; the tenth polypeptide is hrp24; and the eleventh polypeptide is sys18.

In a preferred embodiment of the present disclosure, the fusion protein consists of three same and/or different PAMP polypeptides, and optionally, there is at least one linker or no linker between two adjacent PAMP polypeptides.

In a preferred embodiment of the present disclosure, the fusion protein consists of four same and/or different PAMP polypeptides, and optionally, there is at least one linker or no linker between two adjacent PAMP polypeptides.

In a preferred embodiment of the present disclosure, the fusion protein consists of five same and/or different PAMP polypeptides, and optionally, there is at least one linker or no linker between two adjacent PAMP polypeptides.

In a preferred embodiment of the present disclosure, the fusion protein consists of six same and/or different PAMP polypeptides, and optionally, there is at least one linker or no linker between two adjacent PAMP polypeptides.

In a preferred embodiment of the present disclosure, the fusion protein consists of seven different PAMP polypeptides, and optionally, there is at least one linker or no linker between two adjacent PAMP polypeptides.

Preferably, the fusion protein consists of seven different PAMP polypeptides and at least 6 linkers. Preferably, the seven different PAMP polypeptides are selected from any combination of seven of flg22, nlp20, elf18, pip1, pep1, csp22, flgII-28, ralf17, pep13, hrp24 and sys18.

An arrangement order of the seven different PAMP polypeptides and the specific types of the linkers are not specifically defined herein, and proper adjustments may be made according to the types of plants or types of targeting pathogenic microorganisms, as along as the immune resistance of plants can be induced. Moreover, the fusion proteins with more immune epitopes, better effect and lower use cost, which are obtained through adjustment and optimization, all fall within the protection scope of the present disclosure.

The fusion protein usually requires to be subjected to fusion expression with a protein tag together. The protein tag refers to a kind of polypeptide subjected to fusion expression with a target protein together by using DNA recombination in vitro for convenience in expression, detection and purification of the target protein.

The protein tag, to which the fusion protein of the present disclosure may be linked, includes, but not limited to, HIS, GST, Flag, MBP, HA, c-Myc, eGFP, eYFP and eCFP.

In a preferred embodiment of the present disclosure, the fusion protein consisting of the seven different PAMP polypeptides, which are flg22, nlp20, elf18, pip1, pep1, csp22 and flgII-28, comprises an amino acid sequence shown as SEQ ID NO: 15.

The amino acid sequence shown as SEQ ID NO: 15 shows that the fusion protein is linked to an HIS protein tag, and the arrangement order of the seven different PAMP polypeptides are elf18, csp22, flg22, flgII-28, nlp20, pep1 and pip1 in sequence. There are linkers between the adjacent PAMP polypeptides, and the linkers select GAG and AGA.

In a preferred embodiment of the present disclosure, the fusion protein consisting of the seven different PAMP polypeptides flg22, nlp20, elf18, pip1, pep1, csp22 and flgII-28, includes an amino acid sequence, which is a functional homologous sequence having at least 80% sequence identity to the amino acid sequence shown as SEQ ID NO: 15.

The functional homologous sequence with the identity includes, but is not limited to, a functional homologous sequence having 80% or above, 85% or above, 90% or above, 95% or above, 98% or above or 99% or above sequence identity to the amino acid sequence shown as SEQ ID NO: 15.

The fusion protein consisting of the seven different PAMP polypeptides uses a suitable linker and a tag protein in combination. Compared with a single PAMP polypeptide, the assembled fusion protein having more immunocompetent epitopes may more rapidly and sensitively bind to the receptors on surfaces of the plant cells, rapidly induce defense immune responses of plants, resist pathogenic microorganisms and improve disease resistance of plants when being used in the plants, and further has the advantages of low toxicity, short degradation period, high environment compatibility and the like.

In the second aspect of the present disclosure, provided is a nucleotide sequence coding the fusion protein.

It should be noted that, due to variable constitution of the fusion protein, there is at least one linker or no linker between two adjacent PAMP polypeptides, and the number and the type of the linker are variable. Therefore, correspondingly, the nucleotide sequence coding the fusion protein is also variable. Hence, an arrangement order and the number of bases in the nucleotide sequence are not specifically limited herein. The nucleotide sequence capable of coding the fusion protein and a complementary sequence, a degenerate sequence or a homologous sequence of the nucleotide sequence all fall within the protection scope of the present disclosure.

In a preferred embodiment of the present disclosure, the nucleotide sequence coding the fusion protein having the amino acid sequence shown as SEQ ID NO: 15 is shown as SEQ ID NO: 16.

In a preferred embodiment of the present disclosure, the nucleotide sequence coding the fusion protein having the amino acid sequence shown as SEQ ID NO: 15 hybridizes, under the stringent conditions, to the nucleotide sequence shown as SEQ ID NO: 16 and can code the fusion protein.

Exemplarily, the “stringent conditions” described herein refers to the conditions that a probe hybridizes to its target sequences to the detectable degree exceeding that to which the probe hybridizes to other sequences (for example, at least two times of the background). The stringent condition is sequence-dependent and variable in different environments. By controlling the stringency of the hybridization and/or washing conditions, target sequences that are 100% complementary to the probe may be identified.

In a preferred embodiment of the present disclosure, the nucleotide sequence coding the fusion protein having the amino acid sequence shown as SEQ ID NO: 15 is a degenerate sequence of the nucleotide sequence shown as SEQ ID NO: 16. The degenerate sequence refers to that, after a certain or more nucleotide sequences in the nucleotide sequence shown as SEQ ID NO: 16 are changed, the type of amino acid sequences correspondingly coded by the changed position of the nucleotide sequence is unchanged, and the amino acid sequence of the coded fusion protein is unchanged.

In a preferred embodiment of the present disclosure, the nucleotide sequence for coding the fusion protein having the amino acid sequence shown as SEQ ID NO: 15 is a homologous sequence of the nucleotide sequence shown as SEQ ID NO: 16. Preferably, the homologous sequence is a polynucleotide sequence having at least 85% or above sequence identity to the nucleotide sequence shown as SEQ ID NO: 16.

The homologous sequence includes, but is not limited to, polynucleotide sequences having 85% or above, 88% or above, 90% or above, 93% or above, 95% or above, 98% or above or 99% or above sequence identity to the amino acid sequence shown as SEQ ID NO: 16, and capable of coding the fusion protein.

In the third aspect of the present disclosure, provided is a vector, into which the nucleotide sequence coding the fusion protein is introduced.

The specific type of the vector is not limited. A recombinant expression vector can be constructed by linking the nucleotide sequence to the vector successfully. The fusion protein can be normally expressed by the expression vector in host cells.

Furthermore, the vector includes, but is not limited to, pET-28b(+), pETBlue-1, pETBlue-2, pET-32, pET-34b(+), pET-35b(+), pET-30 EK/LIC, pET-32 EK/LIC, pET-34 EK/LIC and pET-36 EK/LIC. In the fourth aspect of the present disclosure, provided is a microorganism or cell, into which the nucleotide sequence coding the fusion protein and/or the vector are introduced.

It should be noted that the “and/or” herein may be explained as: 1. the microorganism or cell individually includes the nucleotide sequence coding the fusion protein; 2. the microorganism or cell individually contains the vector, and the vector includes the nucleotide sequence coding the fusion protein; 3. the microorganism or cell includes the nucleotide sequence coding the fusion protein and contains the vector at the same time, and the vector includes the nucleotide sequence coding the fusion protein at the same time. The microorganism may be any prokaryotic cell or eukaryotic cell capable of normally expressing the fusion protein.

In a preferred embodiment, the microorganism or cell refers to a particular microorganism or cell into which the nucleotide sequence coding the fusion protein is introduced, including a progeny, carrying the vector of such microorganism.

In a preferred embodiment, the microorganism includes one or more of Escherichia coli, Agrobacterium or Bacillus subtilis; and preferably, Escherichia coli.

Furthermore, Escherichia coli includes, but is not limited to, the following strains: BL21(DE3), λDE3, Rosetta™, K-12, HMS174, NovaBlue, Tuner and OrigamiB.

Furthermore, Agrobacterium includes, but is not limited to, the following strains: EH101, EHA105, C58C1 and LBA4404.

Furthermore, Bacillus subtilis includes, but is not limited to, the following strains: pMA5, PUB110, pE194 and pWB.

Furthermore, inside or outside the plants, methods for transforming the above nucleotide sequence coding the fusion protein and/or the above vector into a host microorganism include, but are not limited to, thermal activation, heat shock, electroporation, calcium phosphate precipitation, polyethylene glycol (PEG) transformation, lipid transfection and microinjection.

In the fifth aspect of the present disclosure, provided is a plant immune inducer containing the fusion protein, the vector or the microorganism or cell.

The plant immune inducer refers to an exogenous organism or molecule capable of inducing or activating the immune responses of the plants and improving the resistance of the plants to certain pathogenic microorganisms. In the present disclosure, the fusion protein, the vector including the nucleotide sequence coding the fusion protein or the microorganism may serve as a raw material for preparing the plant immune inducer. The plant immune inducer is then applied to agricultural production as a bio-pesticide product, providing advantages of low use concentration and being effective quickly and the advantages of low cost, low toxicity, short degradation period, high environment compatibility and the like.

Further, the plant immune inducer further includes one or more of agronomically acceptable vectors, excipients, diluents or solvents.

In a process for preparing the plant immune inducer, in addition to the fusion protein, the vector or the microorganism, serving as a main material, various agronomically acceptable vectors, excipients, diluents or solvents, serving as an excipient, are required to obtain more forms of the plant immune inducer with more stable effect and more convenience in use.

Furthermore, the plant immune inducer is in a form selected from the group consisting of a powder, a soluble powder, a wettable powder, a granule, an aqueous solution, a microemulsion, a suspension and a water dispersible granule. The plant immune inducers are of a variety of forms; and may be used in a wider range, so that the plant immune inducer may be better applied to different varieties of plants.

In the sixth aspect of the present disclosure, provided is a method for preparing the fusion protein, including the step of cultivating the microorganism or cell containing the nucleotide or the step of artificially synthesizing the fusion protein.

Preferably, the method includes the following steps of:

(a) synthesizing the nucleotide sequence, and preferably, before the synthesizing, analyzing, designing and assembling the nucleotide sequence coding the fusion protein;

(b) transforming the synthesized nucleotide sequence (preferably, through the vector) into the microorganism or cell, and cultivating the microorganism or cell to express the fusion protein; and

(c) optionally, collecting and purifying the expressed fusion protein.

By using the method for preparing the fusion protein, with technical combined use of the PTI mechanism and gene engineering, a new recombinant fusion protein is constructed by at least three same and/or different PAMP polypeptides, which have already been discovered, through the gene engineering technology, and the multi-immune-epitope fusion protein which does not exist in nature is obtained through a protein expression technology. The method is simple in preparation process, short in consumed time and low in cost and effectively solves the problem that preparation of plant immune PAMP polypeptides cannot be applied to agricultural production in a long time due to high production cost.

In a preferred embodiment, the method for preparing the fusion protein includes the following steps of:

(a) selecting nucleotide sequences of seven different PAMP polypeptides; analyzing, designing and assembling the nucleotide sequences into the nucleotide sequence, shown as SEQ ID NO: 16; coding the fusion protein online by using a bioinformatics software Geneious R9 in combination with a Swiss-model, and artificially synthesizing the nucleotide sequence; where the seven different PAMP polypeptides are flg22, nlp20, elf18, pip1, pep1, csp22, flgII-28 respectively;

(b) after linking the synthesized nucleotide sequence to a pET-28b(+) expression vector by using a gene engineering method, transforming the pET-28b(+) expression vector into Escherichia coli BL21(DE3) for induced expression of the fusion protein;

(c) collecting and purifying the expressed fusion protein; and

(d) by sequencing the expressed fusion protein, discovering that the fusion protein includes the amino acid sequence shown as SEQ ID NO: 15.

In the seventh aspect of the present disclosure, provided is a use of the fusion protein, or the plant immune inducer or the fusion protein prepared by the method for preparing the fusion protein for improving the disease resistance of plants, inducing plant defense responses and/or resisting pathogenic microorganisms.

Furthermore, the plants include, but are not limited to, Arabidopsis, corns, wheat, rice, tomatoes and tobaccos.

Furthermore, the pathogenic microorganisms include, but are not limited to, Pseudomonas syringae, Fusarium graminearum, Magaporthe grisea and a tobacco mosaic virus.

In the specific use process, the fusion protein or the plant immune inducer may be applied to plants. However, the specific application method and application amount are not limited and may be reasonably selected according to the varieties of the plants and the types of diseases and insect pests.

The fusion protein and/or the plant immune inducer may be interacted with receptor proteins on cell membranes of the plants, and thus the defense responses of plants can be rapidly triggered to resist infection of the pathogenic bacteria. Therefore, the fusion protein may be applied to agricultural production as a bio-pesticide product to activate intrinsic disease resistance of plants, improving the ability of the plants to resist the pathogenic microorganisms, and reducing the use of a chemical pesticide.

The technical solution of the present disclosure has the following beneficial effects:

(1) With one or more immune epitopes, the fusion protein provided by the present disclosure may be applied in a low concentration, act rapidly, and rapidly and widely bind to receptors on surfaces of plant cells, providing a wide spectrum and high efficiency, inducing immune responses of various plants, weakening infection ability of pathogenic microorganisms, and substantially improving the disease resistance of plants.

(2) Compared with the PAMP polypeptides, the fusion protein provided by the present disclosure may be prepared in a low cost, substantially reducing agricultural production cost when being used in agriculture as a bio-pesticide product.

(3) The fusion protein provided by the present disclosure cause no disease resistance of plants, providing advantages of low toxicity, short degradation period, high environment compatibility and the like.

(4) The method for preparing the fusion protein provided by the present disclosure has a simple process, short time consumption and low economic cost input, effectively solveing the problem that preparation of plant immune PAMP polypeptides cannot be applied to agricultural production in a long time due to high production cost.

### BRIEF DESCRIPTION OF THE DRAWINGS

FIG. 1 is an SDS-PAGE electrophoretogram showing a heptapeptide fusion protein His-MP7.

FIG. 2 is a diagram showing accumulation of callose in Arabidopsis plants treated with different concentrations of fusion protein His-MP7 and a PAMP polypeptide flg22.

FIG. 3 is a diagram showing accumulation of callose in Arabidopsis plants treated with 100 nM fusion protein His-MP7 and different PAMP polypeptides.

FIG. 4 is a diagram showing production of reactive oxygen species (ROS) induced by a fusion protein His-MP7 in a corn plant.

FIG. 5 is a diagram showing an experimental result that a fusion protein His-MP7 enhances the resistance of an Arabidopsis plant to DC3000 pathogenic bacteria.

FIG. 6 is a diagram showing an experimental result that a fusion protein His-MP7 enhances the resistance of a corn plant to Fusarium graminearum, wherein “a” shows actual growth comparison on inhibition of Fusarium graminearum infection in corn by the fusion protein His-MP7; and “b” is an experimental result histogram of inhibiting Fusarium graminearum infection in corn by the fusion protein His-MP7.

FIG. 7 is a diagram showing an experimental result that a fusion protein His-MP7 enhances the resistance of a rice plant to Magaporthe grisea, wherein “a” shows actual growth comparison on inhibition of Magaporthe grisea infection in rice by the fusion protein His-MP7; and “b” is an experimental result histogram of inhibiting Magaporthe grisea infection in rice by the fusion protein His-MP7.

### DETAILED DESCRIPTION OF THE INVENTION

In the present disclosure, the term “PAMP polypeptide” refers to a relatively conserved polypeptide fragment, having the immune activation ability, in a sequence of a protein molecule, which is found through contrastive analysis on homologous sequences in intrinsic proteins of various pathogenic fungi or bacteria or plants and usually serves as a signaling molecule polypeptide for perceiving infection of pathogenic bacteria.

In the present disclosure, the term “linker” refers to having at least one amino acid residue, preferably, at least two consecutive amino acid residues.

In the present disclosure, the term “plant immune inducer” refers to an exogenous organism or molecule capable of inducing or activating immune responses of the plants and improving the resistance of the plants to certain pathogenic microorganisms.

In the present disclosure, the term “PTI mechanism” is named as pathogen-associated molecular pattern-triggered immunity (PAMP-Triggered Immunity) mechanism, referring to a mechanism that the immune responses of the plants are activated after the PAMP signaling molecule is recognized by a plant cell receptor.

In the present disclosure, “protein tag” refers to a kind of polypeptide subjected to fusion expression with a target protein together by using DNA recombination in vitro for convenience in expression, detection and purification of the target protein.

The technical solutions in the examples of the present disclosure are described clearly and completely in the following with reference to accompanying figures in the examples of the present disclosure. Apparently, the described examples are only part rather than all of the examples of the present disclosure. Based on the examples of the present disclosure, all the other examples obtained by those of ordinary skill in the art without inventive effort are within the scope of the present disclosure.

In the following examples, there are introductions for sources of a part of plant materials, strains and viruses.

Plant materials: rice (Oryza sativa L.) selected is Nipponbare (NPB) which belongs to short-grain japonica rice (NPB is an international general variety which has been subjected to whole genome sequencing), corns selected are Jundan 20, and the rice and the corns are both purchased from the market; and cultivated tomatoes (Solanum lycopersicum) and a wild-type Arabidopsis col-0 from Columbia and N89 tobacco lines are all from a research group of Professor Yi Cai from College of Life Science in Sichuan Agricultural University.

Strains and viruses: Pseudomonas syringae DC3000, Magaporthe grisea race ZB15, a tobacco mosaic virus and Fusarium graminearum are all from the research group of Professor Yi Cai from College of Life Science in Sichuan Agricultural University.

In addition, materials, reagents, consumables and the like, sources of which are not mentioned in the examples, may all be purchased from the market.

An His tag protein purification kit is purchased from ComWin Biotech Co., Ltd with a catalog number of CW0894; and a BCA protein assay kit is purchased from Solarbio Company with a catalog number of PC0020-500 micropores (50T).

### Example 1 Molecular Design and Nucleotide Sequence Obtaining of Heptapeptide Fusion Protein His-MP7

(1) 7 different PAMP polypeptides: flg22, nlp20, elf18, pip1, pep1, csp22 and flgII-28 were selected from 11 different PAMP polypeptides: flg22, nlp20, elf18, pip1, pep1, csp22, flgII-28, elf18, pep13, ralf17, hrp15 and sys18 and mutants thereof and were assembled into a fusion protein with linkers of AGA and GAG.

(2) Analysis (ProtScale) on molecular weight, amino acid composition (ProtParam) and basic properties of hydrophobicity and the like was conducted on the sequence of the protein by using an online analysis platform ExPASy (https://www.expasy.org); and a structure of the protein was modeled by using a Phyre2 online platform (http://www.sbg.bio.ic.ac.uk/phyre2/html/page.cgi?id=index).

(3) In combination with an analysis result, one design solution was screened out, and the fusion protein was named as MP7, having an amino acid sequence shown as SEQ ID NO: 15.

(4) A nucleotide sequence coding the fusion protein MP7 was designed by using the bioinformatics software Geneious R9 and an online analysis platform Jcat (http://www.jcat.de) to obtain a nucleotide sequence shown as SEQ ID NO: 16. The nucleotide sequence was artificially synthesized.

### Example 2 Expression and Purification of Heptapeptide Fusion Protein His-MP7

(1) A nucleotide sequence shown as SEQ ID NO: 16 was cloned into a pET-28b(+) expression vector (Novagen) at sites HindIII and XhoII. The pET-28b(+) expression vector was heat shocked and transformed into Escherichia coli DH5a. A positive clone colony was selected, shaken, and extracted for plasmids. After being verified through enzyme digestion and sequencing, it was then heat shocked and transformed into Escherichia coli BL21(DE3) to obtain Escherichia coli containing a recombinant plasmid pET-28b-MP7, named as BL21(DE3)/pET-28b-MP7.

(2) BL21(DE3)/pET-28b-MP7 was subjected to induced expression through the following steps. An expression strain was inoculated to an LB liquid medium, and cultured overnight at 37° C. and 200 rpm/min shaking to obtain a first bacterial solution, which was then transferred to an LB liquid medium containing 100 μg/mL kanamycin based on a volume ratio of 1 to 100, and was cultured under shaking continuously at 37° C. and 200 rpm/min, until a concentration OD₆₀₀ nm of the bacterial solution was 0.6. After that, it was added with IPTG with a final concentration of 0.5 mmol/L, cultured under shaking for 12 h at 28° C. and 200 rpm/min to obtain a second bacterial solution. The second bacterial solution was centrifuged at 12000 rpm/min to collect cell pellets, which were then added into a PBS buffer, ultrasonically broken and then centrifuged at 4° C. and 12000 rpm/min. A supernate was then collected.

(3) The supernate was purified with a His tag protein purification kit (soluble protein) through the following specific operation. 5 mL of Ni-Agarose was filled in an empty affinity column. The supernate was slowly flowed through the affinity column. It was then washed by using 6 column volumes of PBS buffer containing 10 mM imidazole to remove impurities. Finally, it was then eluted by using 5 column volumes of PBS buffer containing 500 mM imidazole, and the eluate passing the column was collected. Such eluate was the fusion protein His-MP7 solution.

(4) The concentration of the fusion protein His-MP7 was detected as 0.2 mg/mL by using a BCA protein assay kit. Detected by SDS-PAGE, as shown in FIG. 1, the fusion protein His-MP7 was purified to obtain an expressed protein (His-MP7) containing His tag and having a molecular weight of about 23 kDa.

### Example 3 Study on Titer of Immune Activation of Heptapeptide Fusion Protein His-MP7

In immune responses of plant cells, callose might accumulate. The callose might enhance the mechanical strength of plant cell walls and block channels through which pathogens spread between the cells, thereby limiting invasion of pathogenic microorganisms. With a model plant Arabidopsis as a material and callose accumulation as immune index, the immune activation abilities of different concentrations of fusion proteins His-MP7 were analyzed and were compared to that of a single PAMP polypeptide. A specific experimental operation was as follows:

I. Comparison in Callose Accumulation, Caused By Different Concentrations of Fusion Protein His-MP7, of Arabidopsis

Final concentrations of the fusion protein His-MP7 and a polypeptide flg22 were adjusted to be 1 μM, 100 nM and 10 nM respectively, with water being served as a control. Four-week old Arabidopsis leaves were infiltrated with an injection syringe permeation method for 12 h, the treated leaves were collected and put in a 6-well plate, and a suitable amount of washing buffer was added for 4 h incubation. The washing buffer was substituted by an aniline blue staining solution with a final concentration of 0.1 mg/mL for staining at a room temperature in the dark for 1 h, and the callose accumulation was observed using a 10 times objective lens of a fluorescence microscope. Each treatment contained three biological replicates with three repeated experiments.

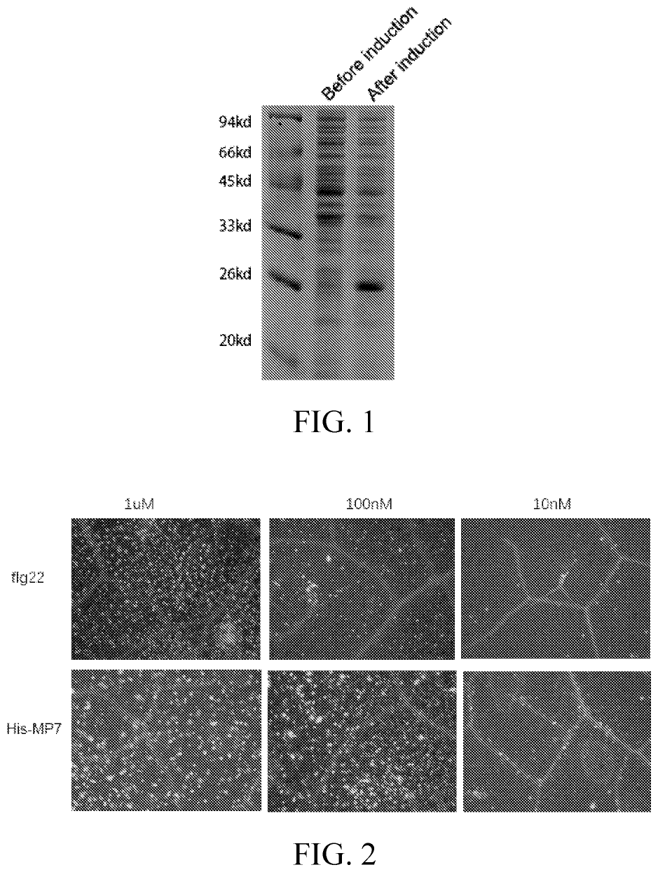

Experimental results were shown in FIG. 2. With 100 nM fusion protein His-MP7, callose accumulation of Arabidopsis could be obviously induced and the intensity was equivalent to that induced by 1 μM PAMP polypeptide flg22.

II. Comparison in Immune Activation Ability Between Fusion Protein His-MP7 and Different Polypeptides Under Same Concentration

A final concentration of the fusion protein His-MP7 and each of polypeptides flg22, nlp20, elf18, pip1, pep1, csp22 and flgII-28 was adjusted to be 100 nM, with csp22 and flgII-28 being served as negative controls. The four-week old Arabidopsis leaves were infiltrated with an injection syringe permeation method; and a sample treatment method was the same as mentioned above.

Experimental results were shown in FIG. 3. The immune activity effect of 100 nM fusion protein His-MP7 was superior to those of all the PAMP polypeptides.

III. Induction of Oxidative Burst Immune Response in Plants with Fusion Protein His-MP7

Oxidative burst was considered as one of earliest responses of plants to pathogenic microorganisms and played an important role in defense responses of the plants. Researches had proved that reactive oxygen species might directly serve as an antimicrobial agent in the plants to result in direct toxicity to the pathogenic microorganisms and inhibition in growth of the pathogenic microorganisms. After the plants were infected by the pathogenic microorganisms, the reactive oxygen species could be produced and accumulate, and oxidative burst could be caused. With plant corns as a material and the reactive oxygen species as an immune index, the immune activation ability of 100 nM fusion protein His-MP7 was analyzed. A specific experimental operation was as follows:

Two-week old corn leaves were taken, the intermediate portion of each leaf was taken, shorn with a length of 5 cm and immersed into 1 μg/mL auxin 6BA solution. It was added with the fusion protein His-MP7 with a final concentration of 100 nM, with an aqueous solution of auxin 6BA being served as a blank control. The intermediate portions were soaked for 48 h, stained for 12 h using 1 mg/mL DAB solution, eluted for 12 h by a washing buffer (ethyl alcohol:acetic acid:glycerin=3:1:1) and observed after being placed for 30 min.

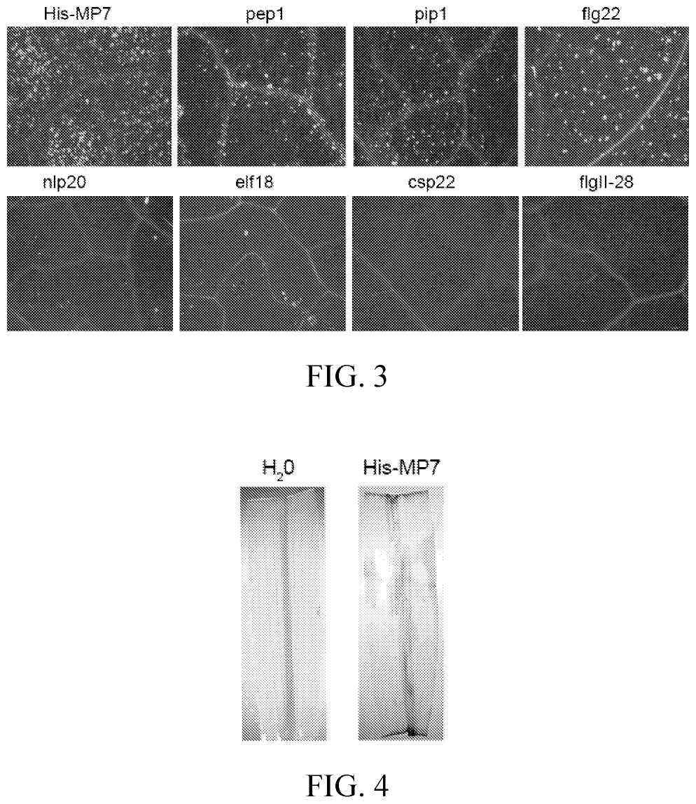

Experimental results were shown in FIG. 4. The 100 nM fusion protein His-MP7 could induce the oxidative burst immune response in the plants.

The above three experiments had proved that plant immune could be efficiently activated with a relatively low concentration of fusion protein His-MP7 (100 nM); and under the same concentration, the immune activation ability of the fusion protein His-MP7 was superior to that of a single PAMP polypeptide.

### Example 4 Detection in Improvement of Disease Resistance of Plants By Heptapeptide Fusion Protein His-MP7

I. A Fusion Protein his-MP7 could Enhance the Resistance of Plants to DC3000 Pathogenic Bacteria.

Experimental Process:

(1) A strain DC3000 of Pseudomonas syringae was inoculated to 20 mL of SOC+str (streptomycin) liquid medium for overnight culture for 14-16 h at 28° C., OD₆₀₀ was determined, and a product was subjected to gradient dilution until OD₆₀₀ was 0.00005 to obtain a bacterial solution.

(2) The fusion protein His-MP7 was added in the bacterial solution until a final concentration was 100 nM, and a bacterial solution, to which water was added, was served as a control. Meanwhile, four-week old healthy Arabidopsis leaves were injected with the bacterial solution and were sampled at Day 0 (four leaves from one plant) and Day 3 (five plants, two leaves from each plant) respectively. Each leaf was punched by a puncher. The punched small discs were taken; 500 μL of 10 mM MgCl₂ was added at Day 0; the small discs were ground in a 1.5 mL EP tube; and 50 μL of each of four samples in the same treatment was spotted on one SOC+str (streptomycin) solid plate (the plate required to be blow-dried, and then the shape of each spotted sample could be kept) and cultured for 16-24 h at 28° C. Samples were photographed and observed for statistics of colony growth.

(3) At Day 3, the two leaves on each plant of the five plants were punched to take small discs similarly; 250 μL of 10 mM MgCl₂ was added; and the small discs were ground in the 1.5 mL EP tube and diluted by MgCl₂ stepwise until 1×10⁻⁵. The five samples were subjected to the same treatment: 10 μL of each diluted sample (for treating 30 samples) was spotted on one SOC+str (streptomycin) solid plate (the plate selected was a square vessel and required to be blow-dried, and then the shape of each spotted sample could be kept) for 16-24 h culture at 28° C., photographed and observed for statistics of colony growth.

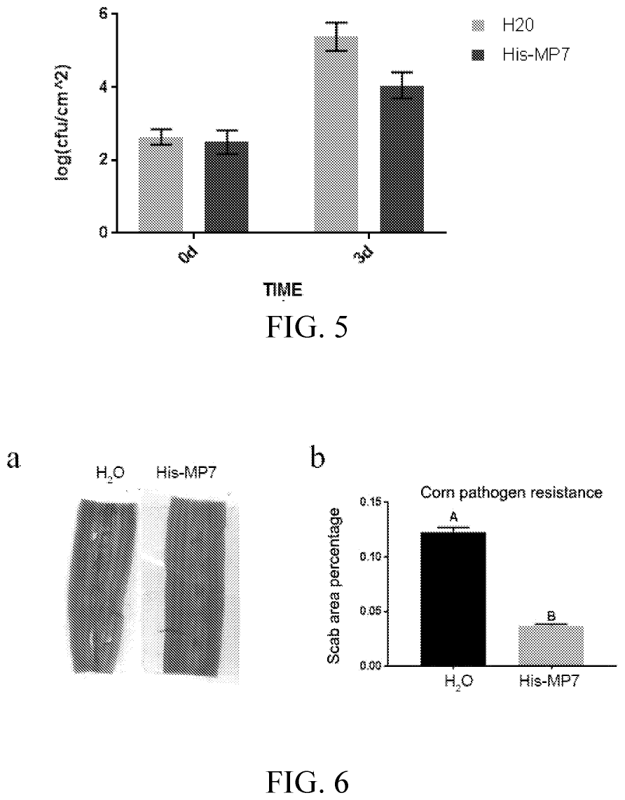

Experimental results were shown as a histogram in FIG. 5. In a control group, a bacterium growth index was 5.38; for Arabidopsis treated by adding 100 nM fusion protein His-MP7, a bacterium growth index was 4.05, and a growth amount of Pseudomonas syringae DC3000 was reduced by 10 or above. Therefore, the His-MP7 could effectively enhance the immunity of the plants to the pathogenic bacteria.

II. A Fusion Protein his-MP7 could Enhance the Resistance of Plants to Fusarium graminearum

Experimental Process:

(1) Mycelia of Fusarium graminearum were inoculated to a CMC liquid medium from the plate for culturing in dark for 3-7 days at 25° C. The resulting product was filtered with gauze, and centrifugated at 10000 rpm/10 min to collect spores. The number of the spores was counted by a blood counting chamber; a concentration of the spores was adjusted to 2×10⁵; and the spores were preserved at 4° C. (used within one month).

(2) Two-week old corn leaves were taken; the intermediate portion of each leaf was taken, shorn with a length of 5 cm and immersed into 1 μg/mL auxin 6BA solution; 12-13 leaves were taken in each treatment; the fusion protein His-MP7 with a final concentration of 100 nM was added; and an aqueous solution of auxin 6BA was served as a control.

(3) Spore liquid of Fusarium graminearum was evenly spotted on each leaf; the leaves were cultured for 3-4 days under the condition of 12-h light and 12-h darkness and at 28° C.; the morbidity was observed; and the scab area percentage was counted by an imageJ software.

Experimental results were shown as growth measurement comparison of infections in a corn leaf, shown as in a, and a histogram shown as b in FIG. 6. For the leaves treated without the fusion protein His-MP7, the scab area percentage was 12.3%; and for the leaves treated with the fusion protein His-MP7, the scab area percentage was 3.6%. It indicated that the fusion protein His-MP7 had significantly enhanced the resistance of the corns to Fusarium graminearum, so that the infection rate of Fusarium graminearum was lowered by 70.8%.

III. A Fusion Protein his-MP7 could Enhance the Resistance of Plants to Magaporthe grisea

Experimental Process:

(1) Magaporthe grisea was inoculated to a CM solid medium for upright standing culture for 13-15 days at 28° C., mycelia were all scraped off by a pipette tip, washed by a small amount of 5-10 mL of sterile water and filtered by a gauze into a 50 mL centrifuge tube; spores were collected through centrifugation at 10000 rpm/10 min; the number of the spores was counted by a blood counting chamber; a concentration of the spores was adjusted to 1×10⁶; and the spores were preserved at a normal temperature (used within one week).

(2) Four-week old rice leaves were taken; the intermediate portion of each leaf was taken, shorn with a length of 5 cm and immersed into 1 μg/mL auxin 6BA solution; 12-13 leaves were taken in each treatment; the fusion protein His-MP7 with a final concentration of 100 nM was added; and an aqueous solution of auxin 6BA was served as a control.

(3) Spore liquid of Magaporthe grisea was evenly spotted on each leaf; the leaves were cultured for 3-4 days under the condition of 12-h light and 12-h darkness and at 28° C.; the morbidity was observed; and the scab area percentage was counted by an imageJ software.

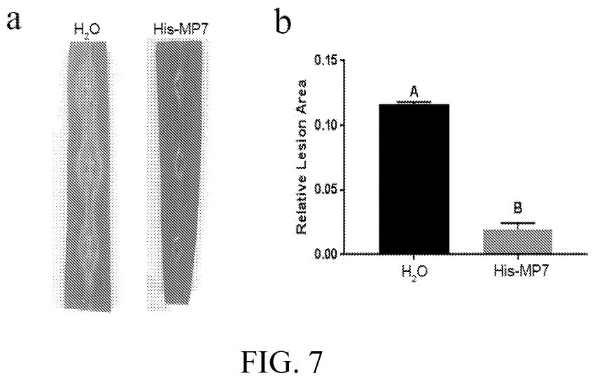

Experimental results of pathogen growth comparison on rice leaves were shown as “a”, and a histogram was shown as “b” in FIG. 7. For the leaves treated without the fusion protein His-MP7, the scab area percentage was 11.6%; and for the leaves treated with the fusion protein His-MP7, the scab area percentage was 1.9%. It indicated that the fusion protein His-MP7 had significantly enhanced the resistance of the rice to Magaporthe grisea; so that the infection rate of Magaporthe grisea had been Lowered by 83.6%, and Harms of Rice Blasts to Rice had been Reduced.

IV. A fusion protein His-MP7 could enhance the resistance of plants to Tobacco mosaic virus (TMV)

The experiment was divided into an experimental group and a control group (with 20 lines of tobacco plants in each group). After the experimental group was injected with a recombinant protein, viruses were inoculated to the experimental group; and after the control group was injected with the sterile water, the viruses were inoculated to the control group. It was inoculated through the following steps:

(1) Fresh leaves infected with TMV were added to a small amount of sterilized phosphate buffer (1:200) and were ground in a mortar and filtered with sterilized gauze to remove the residues of the infected leaves. The above fresh juice was prepared as an inoculum and a concentration of the inoculum of the TMV was adjusted to obtain an aqueous solution of TMV.

(2) When tobacco seedlings were in the 4-5 true leaves period, fully expanded true leaves were selected; a suitable amount of quartz sands were uniformly scattered on the leaf surfaces; and the leaf surfaces were slightly rubbed by an absorbent ball dipped with the aqueous solution of TMV for 1-2 times and then immediately washed with water.

(3) After inoculation for 21 days, the morbidity of tobacco plants was observed. With each plant as a unit for assessment, the grading standard was as follows:

Grade 0: freedom from disease

Grade 1: the base of each interior leaf had a small amount of fading yellow spots along a leaf vein without curling;

Grade 3: newborn leaf had yellow-green stripes parallel to the leaf vein with slightly curling;

Grade 5: newborn leaf had a great amount of chlorotic strips parallel to the leaf vein with a curled, thin and weak leaves;

Grade 7: plant was dwarfed, leaf had yellowish-white strips with curling, and newborn leaves were twisted and prolapsed and could not be normally expanded; and

Grade 9: plant was dwarfed seriously and subjected to chlorina or died.

A disease index and the control effect were computed with the following methods:

Disease index=[Σ(number of infected plants in various grades×relative grade value)/(total number of plants investigated×9)]×100;

Control effect (%)=[(control disease index-treatment disease index)/control disease index]×100.

Results are shown in Table 1 and Table 2:

TABLE 1
Number of Infect Tobacco Plants and Disease Grading

| Unit (plant) | 0 grade | 1 grade | 3 grade | 5 grade | 7 grade | 9 grade |
|---|---|---|---|---|---|---|
| H₂O | 0 | 0 | 2 | 2 | 9 | 7 |
| His-MP7 | 1 | 11 | 6 | 2 | 0 | 0 |

TABLE 2
Disease Index and Control Effect for Tobaccos

| Treatment | Disease index | Control effect |
|---|---|---|
| H₂O | 78.9 | 0 |
| His-MP7 | 21.7 | 72.5% |

Experimental results showed that, for the experiment group treated with the fusion protein His-MP7, the disease index was lowered to 21.7, and the control effect reached 72.5%. The test proved that the fusion protein His-MP7 might enhance the resistance of the tobaccos to TMV.

### Example 5 Molecular Design, Expression and Purification of Various Tripeptide Fusion Proteins

I. Molecular Design of Various Tripeptide Fusion Proteins

(1) 3 PAMP polypeptides were randomly selected from 11 different PAMP polypeptides: flg22, nlp20, elf18, pip1, pep1, csp22, flgII-28, elf18, pep13, ralf17, hrp15 and sys18 and homoeotic mutants thereof and were assembled into various fusion proteins with a linker of AKG, thereby obtaining various design solutions.

(2) Analysis on molecular weight, amino acid composition and basic properties of hydrophobicity and the like was conducted on a sequence of the protein by using an online analysis platform ExPASy; and a structure of the fusion protein was modeled by using a Phyre2 online platform.

(3) In combination with an analysis result, 20 design solutions were screened out and named as TP1, TP2, TP3, TP4, TP5, TP6, TP7, TP8, TP9, TP10, TP11, TP12, TP13, TP14, TP15, TP16, TP17, TP18, TP19 and TP20 respectively. The composition design solutions for various tripeptide fusion proteins were shown in Table 3:

TABLE 3
Design Solutions for Various Tripeptide Fusion Proteins

| Name | Sequence (linker AKG) |
|---|---|
| TP1 | flg22-csp22-pep13 |
| TP2 | flg22-elf18-pep1 |
| TP3 | flg22-elf18-pip1 |
| TP4 | flg22-flg22-flg22 |
| TP5 | flg22-flgII-28-csp22 |
| TP6 | flg22-flgII-28-nlp20 |
| TP7 | flg22-hrp15-sys18 |
| TP8 | flg22ₘ₁-flgII-28ₘ₁-flg22ₘ₂ |
| TP9 | flg22ₘ₁-flg22ₘ₁-flg22ₘ₁ |
| TP10 | flg22ₘ₁-ralf17ₘ₁-csp22ₘ₁ |
| TP11 | flg22ₘ₂-flg22ₘ₂-flg22ₘ₂ |
| TP12 | flg22ₘ₂-nlp20ₘ₁-hrp15 |
| TP13 | flg22-nlp20-csp22 |
| TP14 | flg22-nlp20ₘ₁-pep1 |
| TP15 | flg22-nlp20ₘ₂-csp22ₘ₁ |
| TP16 | flg22-pep1-pip1 |
| TP17 | flg22-ralf17ₘ₁-hrp15 |
| TP18 | flg22-ralf17-hrp24 |
| TP19 | flg22-ralf17-pip1 |
| TP20 | flg22-ralf17-sys18 |

(4) An obtaining method for a nucleotide sequence coding the above 20 tripeptide fusion proteins was the same as that in Example 1.

II. Expression and Purification of Various Tripeptide Fusion Proteins

(1) The obtained nucleotide sequence for conding the above 20 tripeptide fusion proteins was cloned into a pEGX-4T-1 expression vector at sites BamHI and XhoII, the pEGX-4T-1 expression vector was transformed into Escherichia coli DH5a with thermal activation, a positive clone colony was selected for shaking, subjected to plasmid extraction and transformed into Escherichia coli BL21(DE3) with thermal activation after being verified to be correct by digestion and sequencing to obtain Escherichia coli containing a recombinant plasmid pEGX-4T-1-TP, named as BL21(DE3)/pEGX-4T-1-TP.

(2) The Escherichia coli was subjected to induced expression through the following steps. An expression strain was inoculated to an LB liquid medium and cultured overnight at 37° C. and 200 rpm/min shaking to obtain a first bacterial solution which was transferred to an LB liquid medium containing 100 μg/mL ampicillin based on a volume ratio of 1 to 100, and cultured under shaking continuously at 37° C. and 200 rpm/min, until a concentration OD₆₀₀ nm of the bacterial solution was 0.6. After that, it was added with a final concentration of 0.3 mmol/L IPTG, and cultured under shaking for 12 h at 25° C. and 200 rpm/min to obtain a second bacterial solution. The second bacterial solution was centrifuged at 12000 rpm/min to collect cell pellets, which were then added, with a PBS buffer, ultrasonically broken, and centrifuged at 4° C. and 12000 rpm/min. A supernate was collected.

(3) The supernate was purified with a GST tag protein purification kit (soluble protein) to obtain a GST-TP fusion protein solution. The protein solution was quantified by the BCA protein assay kit.

### Example 6 Molecular Design, Expression And Purification of Various Tetrapeptide Fusion Proteins

I. Molecular Design of Various Tetrapeptide Fusion Proteins

(1) 4 PAMP polypeptides were randomly selected from 11 different PAMP polypeptides: flg22, nlp20, elf18, pip1, pep1, csp22, flgII-28, elf18, pep13, ralf17, hrp15 and sys18 and homoeotic mutants thereof and were assembled into various fusion proteins with a linker of AKG. The design methods were the same as those in Example 5. 20 design solutions were screened out and named as FP1, FP2, FP3, FP4, FP5, FP6, FP7, FP8, FP9, FP10, FP11, FP12, FP13, FP14, FP15, FP16, FP17, FP18, FP19 and FP20 respectively. The composition design solutions for various tetrapeptide fusion proteins were shown in Table 4:

TABLE 4
Design Solutions for Various Tetrapeptide Fusion Proteins

| Name | Sequence (linker AKG) |
|---|---|
| FP1 | flg22-csp22-pep13-ralf17 |
| FP2 | flg22-elf18-cap22-nlp20 |
| FP3 | flg22-elf18-pep1-pip1 |
| FP4 | flg22-elf18-pip1-pep1 |
| FP5 | flg22-elf18-nlp20ₘ₂-csp22ₘ₁ |
| FP6 | flg22-flg22-flg22-flg22 |
| FP7 | flg22-flgII-28-csp22-hrp24 |
| FP8 | flg22-flgII-28-nlp20-hrp24 |
| FP9 | flg22-flgII-28-nlp20ₘ₁-csp22ₘ₁ |
| FP10 | flg22-hrp15-sys18-sys18ₘ₁ |
| FP11 | flg22ₘ₁-elf18ₘ₁-nlp20ₘ₂-csp22ₘ₁ |
| FP12 | flg22ₘ₁-flg22ₘ₁-flg22ₘ₁-elf18 |
| FP13 | flg22ₘ₁-nlp20ₘ₂-csp22ₘ₁-pip1ₘ₁ |
| FP14 | flg22ₘ₁-ralf17ₘ₁-csp22-ralf17 |
| FP15 | flg22ₘ₂-flg22ₘ₂-flg22ₘ₂-pip1ₘ₁ |
| FP16 | flg22ₘ₂-nlp20ₘ₁-csp22-hrp15 |
| FP17 | flg22-ralf17ₘ₁-hrp15-flg22 |
| FP18 | flg22-ralf17-pep1-csp22 |
| FP19 | flg22-ralf17-pip1-pep1 |
| FP20 | flg22-ralf17-sys18-hrp15 |

An obtaining method for a nucleotide sequence coding the above 20 tetrapeptide fusion proteins was the same as that in Example 1.

II. Expression and Purification of Various Tripeptide Fusion Proteins, Same as Those in Example 5

### Example 7 Molecular Design, Expression and Purification of Various Pentapeptide Fusion Proteins

I. Molecular Design of Various Tetrapeptide Fusion Proteins

5 PAMP polypeptides were randomly selected from 11 different PAMP polypeptides: flg22, nlp20, elf18, pip1, pep1, csp22, flgII-28, elf18, pep13, ralf17, hrp15 and sys18 and mutants thereof and were assembled into various fusion proteins with a linker of AKG. The design methods were the same as those in Example 5. 20 design solutions were screened out and named as MP5-1, MP5-2, MP5-3, MP5-4, MP5-5, MP5-6, MP5-7, MP5-8, MP5-9, MP5-10, MP5-11, MP5-12, MP5-13, MP5-14, MP5-15, MP5-16, MP5-17, MP5-18, MP5-19 and MP5-20 respectively. The composition design solutions for various pentapeptide fusion proteins were shown in Table 5:

TABLE 5
Design Solutions for Various Pentapeptide Fusion Proteins

| Name | Sequence (linker AKG) |
|---|---|
| MP5-1 | elf18-flg22-csp22-flgII-28-nlp20 |
| MP5-2 | elf18-flg22-flgII-28-pep13-nlp20 |
| MP5-3 | elf18-flg22-flgII-28-pip1-pep1 |
| MP5-4 | elf18-flg22-flgII-28-ralf17-hrp15 |
| MP5-5 | elf18-flg22-flgII-28-ralf17-sys18 |
| MP5-6 | flg22-flg22-flg22-flg22-flg22 |
| MP5-7 | flg22-flgII-28-flg22-flgII-28-flg22 |
| MP5-8 | flg22-flgII-28-nlp20-hrp24-sys18 |
| MP5-9 | flg22-flgII-28-nlp20ₘ₁-csp22ₘ₁-pep13 |
| MP5-10 | flg22-hrp15-sys18-pep13-ralf17 |
| MP5-11 | flg22-flgII-28-nlp20-csp22-pep13 |
| MP5-12 | nlp20-flg22ₘ₁-flg22ₘ₁-flg22ₘ₁-elf18 |
| MP5-13 | nlp20-flg22ₘ₁-nlp20ₘ₂-csp22ₘ₁-pip1 |
| MP5-14 | nlp20ₘ₁-flg22ₘ₁-ralf17ₘ₁-csp22-ralf17 |
| MP5-15 | nlp20ₘ₂-flg22ₘ₂-flg22ₘ₂-flg22ₘ₂-pip1 |
| MP5-16 | nlp20ₘ₁-flg22ₘ₂-nlp20ₘ₁-csp22-hrp15 |
| MP5-17 | flg22-ralf17ₘ₁-hrp15-flg22-csp22 |
| MP5-18 | flgII-28-flg22-ralf17-pep1-csp22 |
| MP5-19 | flgII-28-flg22-ralf17-pip1-pep1 |
| MP5-20 | flgII-28-flg22-ralf17-sys18-hrp15 |

An obtaining method for a nucleotide sequence coding the above 20 pentapeptide fusion proteins was the same as that in Example 1.

II. Expression and Purification of Various Pentapeptide Fusion Proteins, Same as Those in Example 5

### Example 8 Molecular Design of Various Hexapeptide and Heptapeptide Fusion Proteins

6 PAMP polypeptides were randomly selected from 11 different PAMP polypeptides: flg22, nlp20, elf18, pip1, pep1, csp22, flgII-28, elf18, pep13, ralf17, hrp15 and sys18 and mutants thereof and were assembled into various fusion proteins with a linker of AKG. The design methods were the same as those in Example 5. 20 design solutions were screened out and named as MP6-1, MP6-2, MP6-3, MP6-4, MP6-5, MP6-6, MP6-7, MP6-8, MP6-9, MP6-10, MP6-11, MP6-12, MP6-13, MP6-14, MP6-15, MP6-16, MP6-17, MP6-18, MP6-19 and MP6-20 respectively. The composition design solutions for various hexapeptide fusion proteins were shown in Table 6:

TABLE 6
Design Solutions for Various Hexapeptide Fusion Proteins

| Name | Sequence (linker AKG) |
|---|---|
| MP6-1 | elf18-flg22-csp22-flgII-28-nlp20-pep1 |
| MP6-2 | elf18-flg22-flgII-28-csp22-pip1-pep1 |
| MP6-3 | elf18-flg22-flgII-28-nlp20-ralf17-hrp15 |
| MP6-4 | elf18-flg22-flgII-28-pep13-nlp20-pip1 |
| MP6-5 | elf18-flg22-flgII-28-ralf17-sys18-hrp15 |
| MP6-6 | flg22-flg22-flg22-flg22-flg22-flg22 |
| MP6-7 | flg22-flgII-28-flg22-flgII-28-flg22-pep13 |
| MP6-8 | flg22-flgII-28-flg22-ralf17-sys18-hrp15 |
| MP6-9 | flg22-flgII-28-nlp20-csp22-pep13-pep13 |
| MP6-10 | flg22-flgII-28-nlp20-hrp24-sys18-csp22 |
| MP6-11 | flg22-flgII-28-nlp20ₘ₁-csp22ₘ₁-pep13-elf18 |
| MP6-12 | flg22-hrp15-sys18-pep13-ralf17-csp22 |
| MP6-13 | flg22ₘ₁-flgII-28-flg22-ralf17-pep1-csp22 |
| MP6-14 | flg22ₘ₂-flgII-28-flg22-ralf17-pip1-pep1 |
| MP6-15 | nlp20-flg22ₘ₁-flg22ₘ₂-elf18-pep1-nlp20 |
| MP6-16 | nlp20-flg22ₘ₁-nlp20ₘ₂-csp22ₘ₁-pip1-pep1 |
| MP6-17 | nlp20ₘ₁-flg22ₘ₂-nlp20ₘ₁-csp22-hrp24-ralf17 |
| MP6-18 | nlp20ₘ₁-flg22ₘ₁-ralf17ₘ₁-csp22-ralf17-hrp15 |
| MP6-19 | nlp20ₘ₂-flg22ₘ₂-flg22ₘ₂-flg22ₘ₂-pep13-pep1 |
| MP6-20 | ralf17ₘ₁-hrp15-flg22-csp22-pip1-pep13 |

An obtaining method for a nucleotide sequence coding the above 20 hexapeptide fusion proteins was the same as that in Example 1.

II. Expression and Purification of Various Hexapeptide Fusion Proteins, Same as Those in Example 5

### Example 9 Molecular Design, Expression And Purification of Various Heptapeptide Fusion Proteins

7 PAMP polypeptides were randomly selected from 11 different PAMP polypeptides: flg22, nlp20, elf18, pip1, pep1, csp22, flgII-28, elf18, pep13, ralf17, hrp15 and sys18 and mutants thereof and were assembled into various fusion proteins with a linker of AKG. The design methods were the same as those in Example 5. 20 design solutions were screened out and named as MP7-1, MP7-2, MP7-3, MP7-4, MP7-5, MP7-6, MP7-7, MP7-8, MP7-9, MP7-10, MP7-11, MP7-12, MP7-13, MP7-14, MP7-15, MP7-16, MP7-17, MP7-18, MP7-19 and M7-20 respectively. The composition design solutions for various heptapeptide fusion proteins were shown in Table 7:

TABLE 7
Design Solutions for Various Heptapeptide Fusion Proteins

| Name | Sequence (linker AKG) |
|---|---|
| MP7-1 | elf18-flg22-csp22-flgII-28-nlp20-pep1-pip1 |
| MP7-2 | elf18-flg22-flgII-28-csp22-nlp20-pep1-pip1 |
| MP7-3 | elf18-flg22-flgII-28-nlp20-csp22-ralf17-hrp15 |
| MP7-4 | elf18-flg22-flgII-28-nlp20-csp22-hrp24-sys18 |
| MP7-5 | flg22-flgII-28-nlp20-csp22-ralf17-sys18-hrp15 |
| MP7-6 | flg22-flgII-28-nlp20-csp22-pep13-ralf17-hrp15 |
| MP7-7 | flg22-flgII-28-nlp20-csp22-pep13-ralf17-sys18 |
| MP7-8 | flg22-flgII-28-nlp20-csp22-pep13-ralf17-hrp24 |
| MP7-9 | flg22-flgII-28-nlp20-csp22-pep13-ralf17-nlp20 |
| MP7-10 | flg22-flg22ₘ₁-flg22ₘ₂-flg22ₘ₁-flg22ₘ₂- |
| | flg22ₘ₁-flg22ₘ₂ |
| Name | Sequence (linker KRK) |
| MP7-11 | elf18-flg22-csp22-flgII-28-nlp20-pep1-pip1 |
| MP7-12 | elf18-flg22-flgII-28-csp22-nlp20-pep1-pip1 |
| MP7-13 | elf18-flg22-flgII-28-nlp20-csp22-ralf17-hrp15 |
| MP7-14 | elf18-flg22-flgII-28-nlp20-csp22-hrp24-sys18 |
| MP7-15 | flg22-flgII-28-nlp20-csp22-ralf17-sys18-hrp15 |
| MP7-16 | flg22-flgII-28-nlp20-csp22-pep13-ralf17-hrp15 |
| MP7-17 | flg22-flgII-28-nlp20-csp22-pep13-ralf17-sys18 |
| MP7-18 | flg22-flgII-28-nlp20-csp22-pep13-ralf17-hrp24 |
| MP7-19 | flg22-flgII-28-nlp20-csp22-pep13-ralf17-nlp20 |
| MP7-20 | flg22-flg22ₘ₁-flg22ₘ₂-flg22ₘ₁-flg22ₘ₂- |
| | flg22ₘ₁-flg22ₘ₂ |

An obtaining method for a nucleotide sequence coding the above 20 heptapeptide fusion proteins was the same as that in Example 1.

II. Expression and Purification of Various Heptapeptide Fusion Proteins, Same as Those in Example 5

### Example 10 Detection on Immune Responses of Different Fusion Proteins

With a model plant Arabidopsis as a material, callose accumulation as immune index and water as a blank control, all the fusion proteins obtained in Examples 5-9 were adjusted until final concentrations of solutions were all 100 nmol; and four-week old Arabidopsis leaves were infected with an injection syringe permeation method. The specific experimental operation was the same as that in Example 3. Computation was conducted through image processing software ImageJ according to obtained callose accumulation fluorescence images, and the immune activation ability of the fusion proteins to the plants was quantified by virtue of the fluorescence intensity. Computing methods were as follows:

Measured IntDen=Integrated Density=Integrated Optical Density;

Measured Area=Image Area;

MD(mean optical density)=IntDen/Area.

Experimental results are shown in Table 8:

TABLE 8
Identification on Intensity of Immune Responses of Various Fusion Proteins

| Tripeptide | | | Tetrapeptide | | | Pentapeptide | | |
|---|---|---|---|---|---|---|---|---|
| | | Immune | | | Immune | | | Immune |
| Name | MD | activation | Name | MD | activation | Name | MD | activation |
| H₂O | 0.002 | No | H₂O | 0.003 | No | H₂O | 0.002 | No |
| TP1 | 0.058 | Yes | FP1 | 0.035 | Yes | MP5-1 | 0.386 | Yes |
| TP2 | 0.292 | Yes | FP2 | 0.061 | Yes | MP5-2 | 0.235 | Yes |
| TP3 | 0.061 | Yes | FP3 | 0.111 | Yes | MP5-3 | 0.111 | Yes |
| TP4 | 0.089 | Yes | FP4 | 0.356 | Yes | MP5-4 | 0.139 | Yes |
| TP5 | 0.135 | Yes | FP5 | 0.171 | Yes | MP5-5 | 0.097 | Yes |
| TP6 | 0.034 | Yes | FP6 | 0.348 | Yes | MP5-6 | 0.166 | Yes |
| TP7 | 0.358 | Yes | FP7 | 0.051 | Yes | MP5-7 | 0.018 | Yes |
| TP8 | 0.388 | Yes | FP8 | 0.236 | Yes | MP5-8 | 0.295 | Yes |
| TP9 | 0.242 | Yes | FP9 | 0.373 | Yes | MP5-9 | 0.037 | Yes |
| TP10 | 0.378 | Yes | FP10 | 0.156 | Yes | MP5-10 | 0.115 | Yes |
| TP11 | 0.148 | Yes | FP11 | 0.228 | Yes | MP5-11 | 0.278 | Yes |
| TP12 | 0.042 | Yes | FP12 | 0.279 | Yes | MP5-12 | 0.224 | Yes |
| TP13 | 0.193 | Yes | FP13 | 0.223 | Yes | MP5-13 | 0.127 | Yes |
| TP14 | 0.087 | Yes | FP14 | 0.011 | Yes | MP5-14 | 0.195 | Yes |
| TP15 | 0.113 | Yes | FP15 | 0.217 | Yes | MP5-15 | 0.367 | Yes |
| TP16 | 0.146 | Yes | FP16 | 0.163 | Yes | MP5-16 | 0.091 | Yes |
| TP17 | 0.077 | Yes | FP17 | 0.094 | Yes | MP5-17 | 0.098 | Yes |
| TP18 | 0.096 | Yes | FP18 | 0.141 | Yes | MP5-18 | 0.048 | Yes |
| TP19 | 0.160 | Yes | FP19 | 0.044 | Yes | MP5-19 | 0.299 | Yes |
| TP20 | 0.170 | Yes | FP20 | 0.037 | Yes | MP5-20 | 0.301 | Yes |

| Hexapeptide | | | Heptapeptide | | |
|---|---|---|---|---|---|
| | | Immune | | | Immune |
| Name | MD | activation | Name | MD | activation |
| H₂O | 0.003 | No | H₂O | 0.002 | No |
| MP6-1 | 0.396 | Yes | MP7-1 | 0.386 | Yes |
| MP6-2 | 0.359 | Yes | MP7-2 | 0.324 | Yes |
| MP6-3 | 0.114 | Yes | MP7-3 | 0.019 | Yes |
| MP6-4 | 0.065 | Yes | MP7-4 | 0.115 | Yes |
| MP6-5 | 0.266 | Yes | MP7-5 | 0.237 | Yes |
| MP6-6 | 0.382 | Yes | MP7-6 | 0.253 | Yes |
| MP6-7 | 0.317 | Yes | MP7-7 | 0.391 | Yes |
| MP6-8 | 0.250 | Yes | MP7-8 | 0.290 | Yes |
| MP6-9 | 0.051 | Yes | MP7-9 | 0.168 | Yes |
| MP6-10 | 0.116 | Yes | MP7-10 | 0.125 | Yes |
| MP6-11 | 0.071 | Yes | MP7-11 | 0.328 | Yes |
| MP6-12 | 0.386 | Yes | MP7-12 | 0.220 | Yes |
| MP6-13 | 0.288 | Yes | MP7-13 | 0.330 | Yes |
| MP6-14 | 0.221 | Yes | MP7-14 | 0.295 | Yes |
| MP6-15 | 0.348 | Yes | MP7-15 | 0.323 | Yes |
| MP6-16 | 0.123 | Yes | MP7-16 | 0.065 | Yes |
| MP6-17 | 0.314 | Yes | MP7-17 | 0.047 | Yes |
| MP6-18 | 0.023 | Yes | MP7-18 | 0.030 | Yes |
| MP6-19 | 0.090 | Yes | MP7-19 | 0.392 | Yes |
| MP6-20 | 0.102 | Yes | MP7-20 | 0.022 | Yes |

From the experimental results in Table 8, compared with the blank control group, the various tripeptide fusion proteins, the various tetrapeptide fusion proteins, the various pentapeptide fusion proteins, the various hexapeptide fusion proteins, the various heptapeptide fusion proteins and other different fusion proteins provided in Examples 5-9 all had the immune activation ability.

1. A fusion protein, wherein the fusion protein comprises or consists of at least three, four, five, six, seven, or eight same and/or different PAMP (Pathogen-Associated Molecular Pattern) polypeptides; and optionally, there is at least one linker or no linker between two adjacent PAMP polypeptides.

2. The fusion protein according to claim 1, wherein the PAMP polypeptides comprise a first polypeptide for activating an immune receptor FLS2, a second polypeptide for activating an immune receptor RLP23, a third polypeptide for activating an immune receptor EFR, a fourth polypeptide for activating an immune receptor RLK7, a fifth polypeptide for activating an immune receptor PEPR1, a sixth polypeptide for activating an immune receptor CORE1, a seventh polypeptide for activating an immune receptor FLS3, an eighth polypeptide for activating an immune receptor FER, a ninth polypeptide pep13 for activating immune responses of plants, a tenth polypeptide hrp24 and an eleventh polypeptides sys18.

3. The fusion protein according to claim 2, wherein the first polypeptides is flg15 and homoeotic mutants thereof, or flg22 and homoeotic mutants thereof;
the second polypeptides is nlp20 and homoeotic mutants thereof;
the third polypeptides is elf18 and homoeotic mutants thereof;
the fourth polypeptides is pip1 and homoeotic mutants thereof;
the fifth polypeptides is pep1 and homoeotic mutants thereof;
the sixth polypeptides is csp15 and homoeotic mutants thereof, or csp22 and homoeotic mutants thereof;
the seventh polypeptides is flgII-28 and homoeotic mutants thereof;
the eighth polypeptides is ralf17 and homoeotic mutants thereof;
the ninth polypeptides is pep13 and homoeotic mutants thereof;
the tenth polypeptides is hrp15 and homoeotic mutants thereof, or hrp24 and homoeotic mutants thereof; and
the eleventh polypeptides is sys18 and homoeotic mutants thereof.

4. The fusion protein according to claim 3, wherein the first polypeptide is flg22; the second polypeptide is nlp20; the third polypeptide is elf18; the fourth polypeptide is pip1; the fifth polypeptide is pep1; the sixth polypeptide is csp22; the seventh polypeptide is flgII-28; the eighth polypeptide is ralf17; the ninth polypeptide is pep13; the tenth polypeptide is hrp24; and the eleventh polypeptide is sys18.

5. The fusion protein according to claim 1, wherein the fusion protein consists of three same and/or different PAMP polypeptides, and optionally, there is at least one linker or no linker between two adjacent PAMP polypeptides;
preferably, the fusion protein consists of four same and/or different PAMP polypeptides, and optionally, there is at least one linker or no linker between two adjacent PAMP polypeptides;
preferably, the fusion protein consists of five same and/or different PAMP polypeptides, and optionally, there is at least one linker or no linker between two adjacent PAMP polypeptides; and
preferably, the fusion protein consists of six same and/or different PAMP polypeptides, and optionally, there is at least one linker or no linker between two adjacent PAMP polypeptides.

6. The fusion protein according to claim 1, wherein the fusion protein comprises or consists of seven different PAMP polypeptides, and optionally, there is at least one linker or no linker between two adjacent PAMP polypeptides; and preferably, the seven different PAMP polypeptides are selected from any combination of seven of flg22, nlp20, elf18, pip1, pep1, csp22, flgII-28, ralf17, pep13, hrp24 or sys18.

7. The fusion protein according to claim 6, wherein the fusion protein comprises or consists of the following amino acid sequences:
(1) an amino acid sequence shown as SEQ ID NO: 15; or
(2) a functional homologous sequence having at least 80% sequence identity to the amino acid sequence shown as SEQ ID NO: 15.

8. A nucleotide sequence coding the fusion protein according to claim 1.

9. The nucleotide sequence coding the fusion protein according to claim 8, wherein the nucleotide sequence comprises or consists of the following nucleotide sequences:
(1) a nucleotide sequence shown as SEQ ID NO: 16, or
(2) a complementary sequence, a degenerate sequence or a homologous sequence of the nucleotide sequence shown as SEQ ID NO: 16, or
(3) a nucleotide sequence hybridizing, under stringent conditions, to the nucleotide sequence shown as SEQ ID NO: 16 and capable of coding the fusion protein; and
preferably, the homologous sequence is a polynucleotide sequence having at least 85% sequence identity to the nucleotide sequence shown as SEQ ID NO: 16 and coding the fusion protein.

10. A vector, into which the nucleotide sequence according to claim 8 is introduced.

11. A microorganism or cell, into which the nucleotide sequence according to claim 8 is introduced.

12. The microorganism or cell according to claim 11, wherein the microorganism or cell comprises one or more of Escherichia coli, Agrobacterium, Lactobacillus, a yeast or Bacillus subtilis; and preferably, Escherichia coli.

13. A plant immune inducer comprising the fusion protein according to claim 1
preferably, the plant immune inducer further comprises one or more of an agronomically acceptable vector, an excipient, a diluent or a solvent; and
preferably, the plant immune inducer is in a form selected from the group consisting of a powder, a soluble powder, a wettable powder, a granule, an aqueous solution, a microemulsion, suspension and a water dispersible granule.

14. A method for preparing the fusion protein according to claim 1,
the method comprises the following steps of:
(a) synthesizing the nucleotide sequence, and preferably, before the synthesizing, analyzing, designing and assembling the nucleotide sequence coding the fusion protein;
(b) transforming the synthesized nucleotide sequence (preferably, through the vector) into the microorganism or cell, and cultivating the microorganism or cell to express the fusion protein; and
(c) optionally, collecting and purifying the expressed fusion protein.

15. A use of the fusion protein according to claim 1, inducing defense responses and/or resistance against pathogenic microorganisms of plants; preferably, the plants comprise Arabidopsis, corns, wheat, rice, tomatoes and tobaccos; and preferably, the pathogenic microorganisms comprise Pseudomonas syringae, Fusarium graminearum, Magaporthe grisea and a tobacco mosaic virus.


SEQUENCE LISTING (16 sequences)

SEQ ID NO: 1 - protein, 15 aa, Artificial Sequence
  synthesized
  misc_feature: flg15
RINSAKDDAAGLQIA

SEQ ID NO: 2 - protein, 22 aa, Artificial Sequence
  synthesized
  misc_feature: flg2
QRLSTGSRINSAKDDAAGLQIA

SEQ ID NO: 3 - protein, 20 aa, Artificial Sequence
  synthesized
  misc_feature: nlp20
AIMYSWYFPKDSPVTGLGHR

SEQ ID NO: 4 - protein, 18 aa, Artificial Sequence
  synthesized
  misc_feature: elf18
SKEKFERTKPHVNVGTIG

SEQ ID NO: 5 - protein, 13 aa, Artificial Sequence
  synthesized
  misc_feature: pip1
RLASGPSPRGPGH

SEQ ID NO: 6 - protein, 23 aa, Artificial Sequence
  synthesized
  misc_feature: pep1
ATKVKAKQRGKEKVSSGRPGQHN

SEQ ID NO: 7 - protein, 15 aa, Artificial Sequence
  synthesized
  misc_feature: csp15
VKWFNAEKGFGFITP

SEQ ID NO: 8 - protein, 22 aa, Artificial Sequence
  synthesized
  misc_feature: csp22
AVGTVKWFNAEKGFGFITPDDG

SEQ ID NO: 9 - protein, 28 aa, Artificial Sequence
  synthesized
  misc_feature: flgII-28
ESTNILQRMRELAVQSRNDSNSATDREA

SEQ ID NO: 10 - protein, 45 aa, Artificial Sequence
  synthesized
  misc_feature: ralf17
NSIGAPAMREDLPKGCAPGSSAGCKMQPANPYKPGCEASQRCRGG

SEQ ID NO: 11 - protein, 13 aa, Artificial Sequence
  synthesized
  misc_feature: pep13
VWNQPVRGFKVYE

SEQ ID NO: 12 - protein, 15 aa, Artificial Sequence
  synthesized
  misc_feature: hrp15
DLGQLLGGLLQKGLE

SEQ ID NO: 13 - protein, 24 aa, Artificial Sequence
  synthesized
  misc_feature: hrp24
PNQDLGQLLGGLLQKGLEATLQDA

SEQ ID NO: 14 - protein, 18 aa, Artificial Sequence
  synthesized
  misc_feature: sys18
AVQSKPPSKRDPPKMQTD

SEQ ID NO: 15 - protein, 206 aa, Artificial Sequence
  synthesized
  misc_feature: his-MP7
MGSSHHHHHHSSGLVPRGSHMASMTGGQQMGRDPNSSSVDKLSKEKFERTKPHVNVGTIGGAGAVGTVKWFNAEKGFGFITPDDGAGAQRLSTGSRINSAKDDAAGLQIAGAGESTNILQRMRELAVQSRNDSNSATDREAGAGAIMYSWYFPKDSPVTGLGHRAGAATKVKAKQRGKEKVSSGRPGQHNAGARLASGPSPRGRGH

SEQ ID NO: 16 - protein, 621 aa, Artificial Sequence
  synthesized
  misc_feature: his-MP7
ATGGGCAGCAGCCATCATCATCATCATCACAGCAGCGGCCTGGTGCCGCGCGGCAGCCATATGGCTAGCATGACTGGTGGACAGCAAATGGGTCGGGATCCGAATTCGAGCTCCGTCGACAAGCTTAGCAAAGAGAAATTCGAGCGCACCAAACCGCATGTTAACGTTGGTACCATTGGCGGCGCAGGCGCAGTTGGCACCGTTAAATGGTTTAACGCGGAGAAAGGCTTTGGTTTTATTACCCCGGATGATGGCGCAGGCGCACAACGTCTGAGTACCGGTAGCCGCATTAACTCTGCGAAAGATGATGCAGCAGGTCTGCAAATTGCAGGCGCAGGCGAAAGCACCAATATTCTGCAGCGTATGCGCGAACTGGCAGTTCAAAGTCGTAACGATAGTAATTCTGCAACCGATCGCGAAGCAGGTGCCGGCGCAATTATGTATAGCTGGTACTTCCCGAAAGATAGTCCGGTTACCGGTCTGGGTCATCGTGCAGGCGCAGCAACCAAAGTTAAAGCGAAACAGCGCGGCAAAGAAAAAGTCAGTTCTGGTCGTCCGGGTCAACATAACGCAGGCGCACGTCTGGCATCTGGTCCGAGTCCGCGTGGTCGCGGTCATTAA